(19) United States
(12) Patent Application Publication
(10) Pub. No.: US 2024/0161882 A1
(43) Pub. Date: May 16, 2024

(54) System and Method of Electronic Health Record Data Qualification

(71) Applicant: Synerio Technologies, Inc., Fort Worth, TX (US)

(72) Inventor: Ronald Raymond Austríng, English Harbour (AG)

(73) Assignee: Synerio Technologies, Inc., Fort Worth, TX (US)

(21) Appl. No.: 17/775,518

(22) PCT Filed: Apr. 27, 2022
(86) PCT No.: PCT/US2022/026593

§ 371 Date: Nov. 3, 2022

Related U.S. Application Data
(60) Provisional application No. 63/201,388, filed on Apr. 27, 2021.
(60) Provisional application No. 63/201,387, filed on Apr. 27, 2021.
(60) Provisional application No. 63/201,386, filed on Apr. 27, 2021.
(60) Provisional application No. 63/201,383, filed on Apr. 27, 2021.
(60) Provisional application No. 63/201,385, filed on Apr. 27, 2021.

(51) Int. Cl. G16H 10/60

(52) U.S. Cl. CPC G16H 10/60

(57) ABSTRACT

A system and method of electronic health record data qualification is presented. A Qualification List can be used in the practical application of data qualification that uses a predefined list of data to determine if the current values of input fields, which correspond to properties of the predefined list, match any item in the list according to the value of a ‘compare mnemonic’ (e.g., EQ, NE, GT, LT, GE, LE) of the corresponding input field. The system can advantageously override established processing flows by inverting determination values to direct the processing of the acquired data. Advantageously, the system can provide context for the values by retrieving data within an execution context, thereby acting as a gatekeeper to direct subsequent processing.

### TECHNICAL FIELD

The present disclosure generally relates to data qualification systems, and more specifically to systems capable of determining if the current values of input fields match stored values.

### BACKGROUND

In traditional data processing systems applications linearly retrieve data and process the data according to predefined arguments and static processing flows. A typical programming paradigm is to pass information to methods/functions as arguments. This requires that the code calling the function gather all the data required by the function and then pass that data to the function using the arguments of the function. The arguments position, name, and data type, for a specific method/function form what is known as the method/function signature. The calling program must know about the signature of the method/function to be called. This results in a tight coupling between the caller of the function and the function. Further once a function satisfies a threshold condition it must follow the processing path determined for that elected condition.

### SUMMARY

The present disclosure achieves technical advantages as a system and method of electronic health record data qualification. The present disclosure provides for a system integrated into a practical application with meaningful limitations to provide a Qualification List can be used in a system and method of data qualification that uses a predefined list of data to determine if the current values of input fields, which correspond to properties of the predefined list, match any item in the list according to the value of a ‘compare mnemonic’ (e.g., EQ, NE, GT, LT, GE, LE) of the corresponding input field. The system can advantageously override established processing flows by inverting determination values to direct the processing of the acquired data. Advantageously, the system can provide context for the values by retrieving data within an execution context, thereby acting as a gatekeeper to direct subsequent processing.

In one embodiment, a ‘Qualification List’ can be a ‘Property Collection’, with one or more of the following properties:
InputFields—an array of ‘Input Field Property Collection’ references
Qualificationitems—an array of ‘Qualification Item Property Collection’ references.
InvertResult—a ‘Boolean’ field that can indicate whether the result of the qualification should be inverted.
CacheResult—a ‘Boolean’ field that can indicate whether the result of the qualification should be cached for the duration of the ‘execution context’ processing.

The present disclosure solves the technological problem of gathering all the data required by the function and then passing that data to the function using the arguments of the function. The electronic health record data qualification system solves the aforementioned technological problem by providing a dynamic execution function programming paradigm with an addressable universal data location system. Accordingly, the claims herein are necessarily rooted in computer technology as they overcome a problem arising in the realm of computer application execution.

The present disclosure provides a technological solution missing from conventional systems by providing a data qualification system that can pass a ‘function’ a reference to an ‘execution context’ and have the function retrieve the data items it needs through the ‘execution context.’ This approach allows the function itself to be in control of the data items that it needs. If the function requirements change, only the function changes and not the code calling the function. An addressable data locator system called an Execution Context Property Path (XPP) that can access a ‘property’ within a ‘Property Collection,’ a ‘property’ within a graph of nested ‘Property Collections,’ or a ‘property’ within an array of ‘Property Collections.’ In one embodiment, an XPP can be an ascii string consisting of several components to address a ‘property.’ For example, the XPP can be formatted according to the following structure: <property collection><property path><query string>. The XPP can be used in association with a ‘Qualification List.’ Additionally, the ‘Qualification List’ can include a mnemonic that can determine the qualification parameters for data verification. The mnemonic can define how the input field should be compared to the corresponding property value. As such, the qualification lists disclosed herein are data driven, eliminating static programming.

Accordingly, the present disclosure discloses concepts inextricably tied to computer technology such that the present disclosure provides the technological benefit of allowing dynamic data recall to provide a nearly limitless number of inputs that are only provided to the executing function at runtime. The present disclosure technologically surpasses conventional technology by providing a mechanism by which the system knows where to retrieve data prior to runtime and allowing for control logic overrides. Further, the ‘function’ can use a method on the ‘execution context’ to retrieve each required data item using an XPP. In another embodiment, all ‘Property Collections’ that exist in the execution context can be available to the function.

In another embodiment, a ‘Dynamic Function’ is a function that can be loaded at runtime and reloaded if and when a new version of the function becomes available. A ‘Dynamic Function’ can be designed to be called with only one argument, which is a reference to the ‘execution context’. This allows the function to at least:
Read any data items that it needs using an XPP;
Write any data item that it needs using an XPP;
Create, update, or delete ‘Property Collections;’ and
Call all supported ‘methods’ on the ‘execution context.’

One benefit of the application execution environment is how these technology components interact with each other through a common ‘language’ and ‘process methodology.’ The ‘language’ can be the data addressing and access methodology of the ‘execution context’ and an ‘XPP.’ The ‘process methodology’ can use an ‘execution context’ object as the manager of one or more ‘Property Collections,’ such that all data can be created, updated, loaded, and deleted by the application. The ‘execution context’ can also manage the loading of ‘business logic’ in the form of ‘Rules’ and context associated ‘Rule Property’ data through a process of ‘Qualification.’ The ‘Qualification’ process can load ‘Qualification Functions’ and ‘Qualification Lists,’ which have access to all data within the ‘execution context’ and use an XPP to access that data. A ‘Qualification List’ can use the retrieved data to compare against a predefined list of data to determine if there is a match. A ‘Qualification Function’ can use the retrieved data, in conjunction with logic, to determine qualification.

It is an object of the invention to provide a system for electronic health record data qualification. It is a further object of the invention to provide a method of electronic health record data qualification. These and other objects are provided by the present disclosure, including at least the following embodiments.

In one embodiment, a system for electronic health record data qualification can include: a memory storing one or more property collections; and a processor operably coupled to the memory and capable of executing machine-readable instructions to perform program steps, the program steps comprising: receiving a qualification list in a first format from a client, the qualification list including at least one input field array having a property name, a property name value, a mnemonic, and a mnemonic value, an invert result property and an invert result property value; query a database to return matches of a property name value using the mnemonic value; setting a result value to true to indicate that a match was found; determining the invert result value is true and inverting the result value. The program steps further comprising converting the qualification list from the first format into a Property Collection format. The program steps further comprising retrieving a property name value and a mnemonic value from the qualification list for all input field arrays. Wherein the database can be the qualification items array. Wherein the database can be a Property Collection. The program steps further comprising determining a cache result property is False and terminating the qualification. The program steps further comprising determining a cache result property is True and caching the result value as the qualification result. The program steps further comprising returning the result value as the result of the qualification. Wherein the processor addresses data using an execution context property path (XPP). Wherein the at least one input field array also has an XPP property and an XPP value to retrieve the current value of the input field. Wherein the XPP value is an ASCII string.

In another embodiment, a method of electronic health record data qualification can include: receiving, via a processor, a qualification list in a first format from a client, the qualification list including at least one input field array having a property name, a property name value, a mnemonic, and a mnemonic value, an invert result property and an invert result property value; querying a database to return matches of a property name value using the mnemonic value; setting a result value to true to indicate that a match was found; determining the invert result value is true and inverting the result value. Further comprising converting the qualification list from the first format into a Property Collection format. Further comprising retrieving a property name value and a mnemonic value from the qualification list for all input field arrays. Wherein the database can be the qualification items array. Wherein the database can be a Property Collection. Further comprising determining a cache result property is True and caching the result value as a qualification result. Further comprising returning the result value as the result of the qualification. Wherein the processor addresses data using an execution context property path (XPP). Wherein the at least one input field array also has an XPP property and an XPP value to retrieve the current value of the input field.

### BRIEF DESCRIPTION OF THE DRAWINGS

The present disclosure will be readily understood by the following detailed description, taken in conjunction with the accompanying drawings that illustrate, by way of example, the principles of the present disclosure. The drawings illustrate the design and utility of one or more exemplary embodiments of the present disclosure, in which like elements are referred to by like reference numbers or symbols. The objects and elements in the drawings are not necessarily drawn to scale, proportion, or precise positional relationship. Instead, emphasis is focused on illustrating the principles of the present disclosure.

FIG. 1 illustrates an electronic health record data qualification system schematic, in accordance with one or more exemplary embodiments of the present disclosure;

FIG. 2 illustrates a qualification list diagram, in accordance with one or more exemplary embodiments of the present disclosure; and

FIG. 3 illustrates a flowchart exemplifying electronic health record data qualification process flow control logic, in accordance with one or more exemplary embodiments of the present disclosure.

### DETAILED DESCRIPTION

The disclosure presented in the following written description and the various features and advantageous details thereof, are explained more fully with reference to the non-limiting examples included in the accompanying drawings and as detailed in the description. Descriptions of well-known components have been omitted to not unnecessarily obscure the principal features described herein. The examples used in the following description are intended to facilitate an understanding of the ways in which the disclosure can be implemented and practiced. A person of ordinary skill in the art would read this disclosure to mean that any suitable combination of the functionality or exemplary embodiments below could be combined to achieve the subject matter claimed. The disclosure includes either a representative number of species falling within the scope of the genus or structural features common to the members of the genus so that one of ordinary skill in the art can recognize the members of the genus. Accordingly, these examples should not be construed as limiting the scope of the claims.

A person of ordinary skill in the art would understand that any system claims presented herein encompass all of the elements and limitations disclosed therein, and as such, require that each system claim be viewed as a whole. Any reasonably foreseeable items functionally related to the claims are also relevant. Pursuant to Section 904 of the Manual of Patent Examination Procedure, the Examiner, after having obtained a thorough understanding of the invention disclosed and claimed in the nonprovisional application has searched the prior art as disclosed in patents and other published documents, i.e., nonpatent literature. Therefore, as evidenced by the issuance of this patent, the prior art fails to disclose or teach the elements and limitations presented in the claims as enabled by the specification and drawings, such that the presented claims are patentable under 35 U.S.C. §§ 101, 102, 103, and 112.

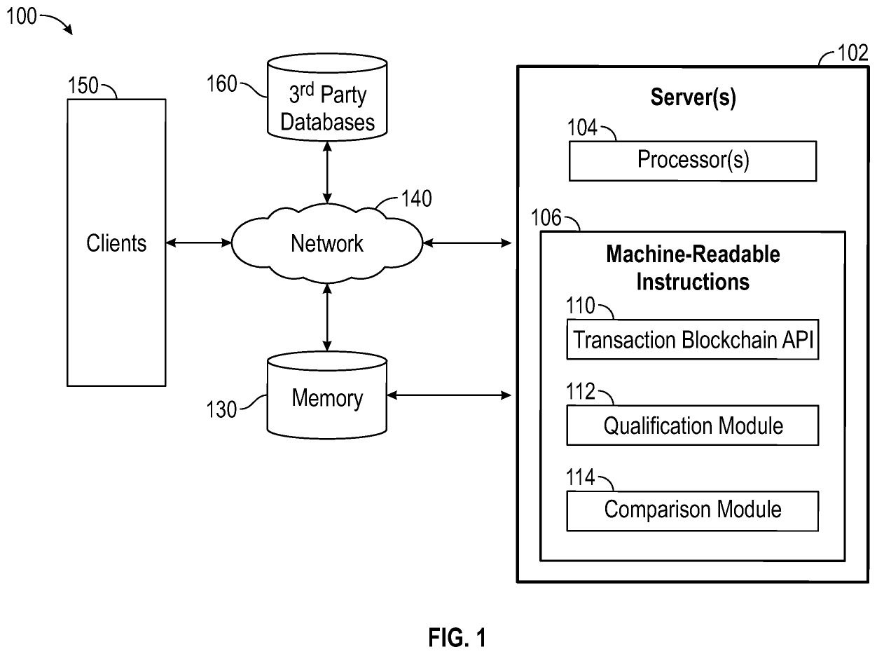

FIG. 1 illustrates an electronic health record data qualification system 100 schematic, in accordance with one or more exemplary embodiments of the present disclosure. The system 100 can include one or more servers 102 having one or more processors 104, a memory 130, machine readable instructions 106, including transaction blockchain API 110, a qualification module 112, and a comparison module 114, among other relevant modules. The server 102 can be operably coupled to one or more clients 150 via a network 140. The clients 150 can be a physical device (e.g., mobile phone, laptop, tablet, desktop computer, wearable device, or other suitable device), program, or application. In another exemplary embodiment, a client 150 can include a mobile phone having a mobile application configured to communicate with the server 102 over the network 140.

In one embodiment, the transaction blockchain API 110 can be provided by the system 100 for accessing the blockchain, the transaction blockchain API 110 having an interface that can define interactions between multiple components. For example, the transaction blockchain API 110 can define the kinds of calls or requests that can be made, how to make them, the data formats that should be used, the conventions to follow, and other suitable functionality related to a blockchain. In another embodiment, the blockchain API 110 can access and retrieve Property Collections. In another embodiment, the transaction blockchain API 110 can address and access data using an XPP, including data on the blockchain.

In one embodiment, a qualification module 112 can determine which business logic (rules) should be executed, and the ‘property values’ to use when executing the rules. This determination can be based on any data contained in a ‘Qualification List’ ‘Property Collection,’ such as: the ‘input data’ received from the client, ‘application configuration data’, ‘listener configuration data’, ‘entities’ (e.g., Patient, Pharmacy, Drug, etc.) that are loaded based on data in the current context, etc. In another embodiment, the qualification module 112 can address and access data using an XPP.

An XPP can provide for data access and addressing of one or more data elements in the system in a reliable and standardized way. In one embodiment, an XPP can be a modified form of a Uniform Resource Identifier (URI) used to address and access a ‘property’ within a ‘Property Collection’ being managed by the ‘Execution Context.’ An XPP can be used to locate a specific ‘Property Collection’ and access a ‘property’ within that collection. In another embodiment, an XPP can be an ascii string. For example, an XPP can consists of several components used to address a ‘property’: <property collection><property path><query string>. In ASCII string form, the XPP can be stored as data and provided from a source external to the application. Particularly, an XPP as an ASCII string (with the proper encoding) can be used to communicate the location of any single piece of data or a collection of data within an application program. Accordingly, the XPP for a particular data structure can be copied, pasted, printed, emailed, sent in an instance messaging program, included as part of the content exposed by another XPP, etc. In another embodiment, An XPP can be used within an ‘execution context’ to access a ‘property’ within a ‘Property Collection’, a ‘property’ within a graph of nested ‘Property Collections,’ or a ‘property’ within an array of ‘Property Collections.’ A ‘Qualification List’ is an example of how an XPP can be stored within external data but loaded into an application and used to retrieve a specific data item from a known ‘Property Collection,’ as discussed in more detail herein. In another embodiment, the XPP can be used to address and access data.

In one embodiment, a comparison module 114 can access one or more fields of a ‘Qualification List’ to determine the mnemonic and relevant comparison data. In another embodiment, the comparison module 114 can use an XPP to access the ‘Qualification List’ and retrieve the associated data at runtime. In another embodiment, the comparison module 114 can then use the data retrieved from the ‘Qualification List’ along with data retrieved from one or more ‘Property Collections’ and compare the data according to the mnemonic.

The aforementioned systems, components, and modules can be communicably coupled to each other via the network 140, such that data can be transmitted therebetween. The network 140 can be the Internet, intranet, computer bus, or other suitable network. The data transmission can be encrypted, unencrypted, over a VPN tunnel, or other suitable communication means. The network 140 can be a WAN, LAN, PAN, or other suitable network type. The network communication can be encrypted using PGP, Blowfish, Twofish, AES, 3DES, HTTPS, or other suitable encryption. The system 100 can be configured to provide communication via the various systems, components, and modules disclosed herein via an application programming interface (API), PCI, PCI-Express, ANSI-X12, Ethernet, Fiber, Wi-Fi, Bluetooth, or other suitable communication protocol or medium. Additionally, third party systems and databases 160 can be operably coupled to the system components via the network 140.

The data transmitted to and from the components of system 100 (e.g., the server 102, memory 130, and clients 150), can include any format, including the XPP format disclosed herein, JavaScript Object Notation (JSON), TCP/IP, XML, HTML, ASCII, SMS, CSV, representational state transfer (REST), or other suitable format. The data transmission can include a message, flag, header, header properties, metadata, and/or a body, or be encapsulated and packetized by any suitable format having same.

The server(s) 102 can be implemented in hardware, software, or a suitable combination of hardware and software therefor, and may comprise one or more software systems operating on one or more servers, having one or more processors 104, with access to memory 130. Server(s) 102 can include electronic storage, one or more processors, and/or other components. Server(s) 102 can include communication lines, connections, and/or ports to enable the exchange of information via a network 140 and/or other computing platforms. Server(s) 102 can also include a plurality of hardware, software, and/or firmware components operating together to provide the functionality attributed herein to server(s) 102. For example, server(s) 102 can be implemented by a cloud of computing platforms operating together as server(s) 102, including Software-as-a-Service (SaaS) and Platform-as-a-Service (PaaS) functionality. Additionally, the server(s) 102 can include memory 130, locally attached, network attached, or both.

Memory 130 can comprise electronic storage that can include non-transitory storage media that electronically stores information. The electronic storage media of electronic storage can include one or both of system storage that can be provided integrally (e.g., substantially non-removable) with server(s) 102 and/or removable storage that can be removably connectable to server(s) 102 via, for example, a port (e.g., a USB port, a firewire port, etc.) or a drive (e.g., a disk drive, etc.). Electronic storage may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage may include one or more virtual storage resources (e.g., cloud storage, a virtual private network, and/or other virtual storage resources). The electronic storage can include a database, or public or private distributed ledger (e.g., blockchain). Electronic storage can store machine-readable instructions 106, software algorithms, control logic, data generated by processor(s), data received from server(s), data received from computing platform(s), and/or other data that can enable server(s) to function as described herein. The electronic storage can also include third-party databases accessible via the network 140. In one embodiment, memory 130 can be a blockchain implemented on one or more platforms, including BigChainDB, nChain, Ethereum, Hyperledger, R3, Ripple, EOS, or other suitable blockchain platform. The blockchain can be a public blockchain (accessible to the general public) or a private blockchain (accessible only by those parties credentialed for access).

Processor(s) 104 can be configured to provide data processing capabilities in server(s) 102. As such, processor(s) 104 can include one or more of a digital processor, an analog processor, a digital circuit designed to process information, an analog circuit designed to process information, a state machine, and/or other mechanisms for electronically processing information, such as FPGAs or ASICs. The processor(s) 104 can be a single entity or include a plurality of processing units. These processing units can be physically located within the same device, or processor(s) 104 can represent processing functionality of a plurality of devices or software functionality operating alone, or in concert.

The processor(s) 104 can be configured to execute machine-readable instructions 106 or machine learning modules via software, hardware, firmware, some combination of software, hardware, and/or firmware, and/or other mechanisms for configuring processing capabilities on processor(s) 104. As used herein, the term “machine-readable instructions” can refer to any component or set of components that perform the functionality attributed to the machine-readable instructions component 106. This can include one or more physical processors 104 during execution of processor-readable instructions, the processor-readable instructions, circuitry, hardware, storage media, or any other components.

The server(s) 102 can be configured with machine-readable instructions having one or more functional modules. The machine-readable instructions 106 can be implemented on one or more servers 102, having one or more processors 104, with access to memory 130. The machine-readable instructions 106 can be a single networked node, or a machine cluster, which can include a distributed architecture of a plurality of networked nodes. The machine-readable instructions 106 can include control logic for implementing various functionality, as described in more detail below. The machine-readable instructions 106 can include certain functionality associated with the system 100. Additionally, the machine-readable instructions 106 can include a smart contract or multi-signature contract that can process, read, and write data to the memory 130, 3rd party database 160, including a database, distributed ledger, or blockchain.

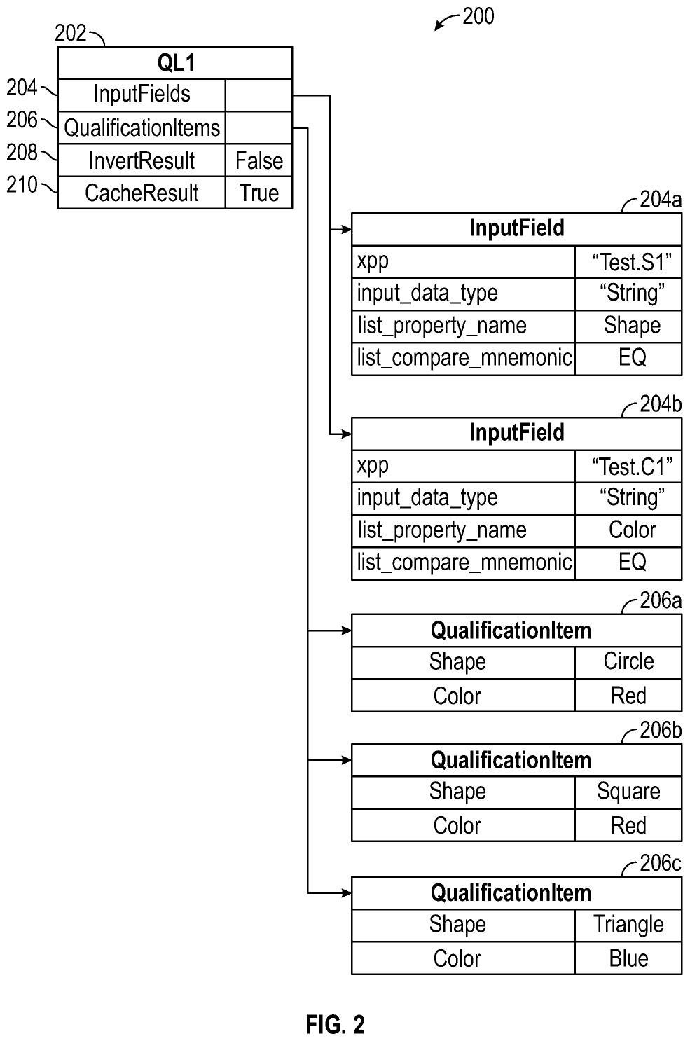

FIG. 2 illustrates a qualification list diagram 200, in accordance with one or more exemplary embodiments of the present disclosure. By way of example, a ‘Qualification List’ can be a ‘Property Collection’ called ‘QL1’ 202, which contains the four properties described above, namely: InputFields 204, QualificationItems 206, InvertResult 208, and CacheResult 210. The ‘InputFields’ property of the ‘QualificationList’ ‘Property Collection’ can be an array of two ‘InputField’ ‘Property Collection’ references. Each ‘InputField’ ‘Property Collection’ can define the following:

| Property Name | Description |
|---|---|
| xpp | The XPP of the input field, which can be used |
| | to retrieve the current value of the input field. |
| input_data_type | This property can define the ‘data type’ of the |
| | input field. Legal values can be (String, Integer, |
| | Decimal, Boolean, Date, Time, DateTime, |
| | ByteArray). |
| list_property_name | This property can define the name of the |
| | corresponding property name within the |
| | ‘QualificationItems’ |
| list_compare_mnemonic | This property can define how the input field |
| | should be compared to the corresponding |
| | property value within the ‘QualificationItems’. |

In one embodiment, as Boolean fields, the ‘InvertResult’ property 208 the ‘CacheResult’ property 210 can be set to ‘True’ or ‘False.’ In another embodiment, the ‘InputField’ property, as an array, can have one or more ‘Input Field Property Collection’ references 204a, 204b. For example, each “InputFields’ reference 204a, 204b can include an ‘xpp’ property, an ‘input_data_type’ property, a ‘list_property_name’ property, and a ‘list_compare_mnemonic’ property, with associated property values. For example, the ‘xpp’ property can be an ASCII string pointing to a data location, such as “Test.S1” or “Test.C2.” The ‘input_data_type’ property can be a data type, such as “String,” or other suitable data type. The list_property_name’ property can have a name, such as “Shape” or “Color,” or other suitable name. The ‘list_compare_mnemonic’ property can list a comparison operator, such as equal (EQ), greater than, less than, greater than or equal to, less than or equal to, not equal to, or other suitable comparison operator. In another embodiment, the ‘QualificationItems’ property, as an array, can have one or more ‘Qualification Item Property Collection’ references 206a, 206b, 206c. For example, each ‘QualificationItems’ reference 206a, 206b, 206c can include one or more entries. The entries can include the ‘list_property_name’ and an associated value for comparison.

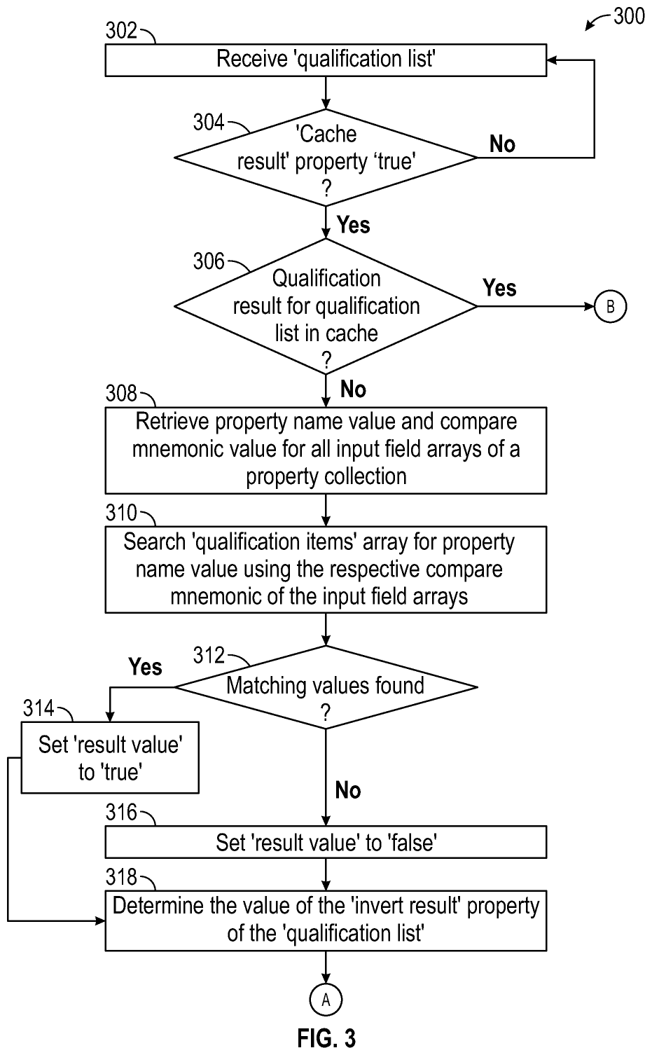

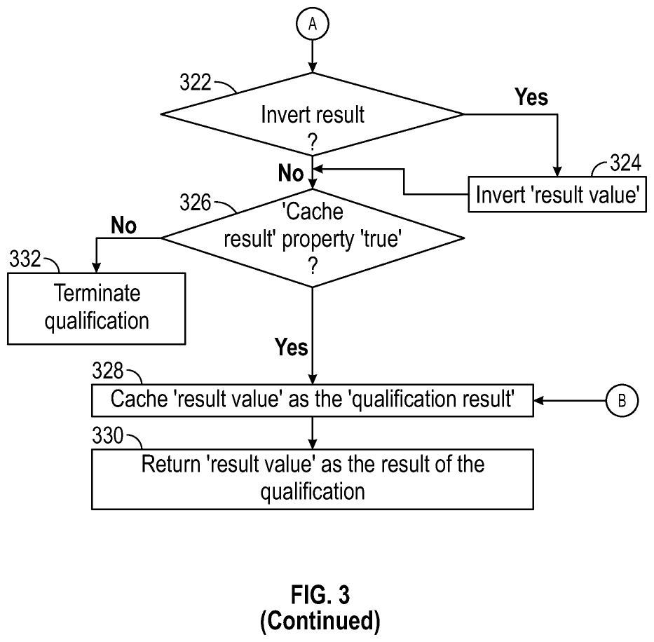

FIG. 3 illustrates a flowchart exemplifying electronic health record data qualification process flow control logic 300, in accordance with one or more exemplary embodiments of the present disclosure. The qualification flow control logic 500 can be implemented as an algorithm on a server 102, a machine learning module, a client 150, a memory 130, a combination of one or more of the aforementioned components, or other suitable system. The qualification process flow control logic 500 can be achieved with software, hardware, an application programming interface (API), a network connection, a network transfer protocol, HTML, DHTML, JavaScript, Dojo, Ruby, Rails, other suitable applications, or a suitable combination thereof.

The qualification process flow control logic 500 can leverage the ability of a computer platform to spawn multiple processes and threads by processing data simultaneously. The speed and efficiency of the qualification process flow control logic 500 can be greatly improved by instantiating more than one process to implement a qualification. However, one skilled in the art of programming will appreciate that use of a single processing thread may also be utilized and is within the scope of the present disclosure.

In one embodiment, a qualification of data can be a certification that data meets that certain criteria provided in a ‘QualificationList.’ After receiving a request from a client 150, the system can qualify it against one or more qualification rules, as listed in the ‘Qualification List.’ If a request qualifies, then additional processing, according to an Execution Context, or otherwise can be conducted. Fundamentally, based upon the outcome of the qualification (all true, portion true, none true, etc.), the request can be dynamically processed, driven by one or more different scenarios based on qualification outcome (e.g., thresholding, deviation, or other suitable metric).

The qualification process flow control logic 300 of the present embodiment begins at step 302, where the control logic 300 can receive a ‘QualificationList.’ The ‘QualificationList’ can include at least one input field array having a property name, a property name value, a mnemonic, and a mnemonic value, an invert result property and an invert result property value. In one embodiment, the ‘QualificationList’ can be from a client. For example, the client can provide a list of properties and the control logic can convert the received list into the Property Collection format and define an InputField. The list can be in one or more different formats (e.g., Oracle®, CSV, text, etc.). The control logic 300 can instantiate an input parser to extract the relevant property fields and associated property values. For example, the input data can be in a format associated with an Oracle® database, a blockchain, a spreadsheet, CSV file, an email, a user interface post form, or other suitable format. The parser can process the ‘QualificationList’ and create and populate a ‘Property Collection’ object, provide a ‘Property Collection Interface’, or otherwise generate a Property Collection to represent or store the received ‘QualificationList’ as a ‘Property Collection.’ The control logic 300 then proceeds to step 304.

At step 304, the control logic 300 can determine whether a ‘CacheResult’ is ‘True.’ For example, the ‘QL1’ ‘Qualification List’ ‘Property Collection’ can be consulted to determine if the ‘CacheResult’ property is ‘True.’ If the ‘CacheResult’ property is ‘True,’ the control logic 300 proceeds to step 306. If the ‘CacheResult’ property is ‘False,’ the control logic 300 returns to step 302.

At step 306, the control logic 300 can determine whether there exists a ‘qualification result’ for the ‘QualificationList’ in cache. For example, if the control logic 300 determines that there is no ‘qualification result’ for the ‘QualificationList’ in cache, processing continues. If there exists a ‘qualification result’ for the ‘QualificationList’ in cache, the control logic 300 proceeds to step 328. If there does not exists a ‘qualification result’ for the ‘QualificationList’ in cache, the control logic 300 proceeds to step 308.

At step 308, the control logic 300 can retrieve property name value and compare mnemonic value for all input field arrays of a property collection. In one embodiment, the control logic 300 can retrieve the property name and the value for all the input field arrays for a particular property collection. The control logic 300 then proceeds to step 310.

At step 310, the control logic 300 can query the ‘QualificationItems’ array for property name value using the respective compare mnemonic of the ‘InputFields’ arrays. The control logic 300 can search the ‘QualificationItems’ array for a property name value using the respective compare to mnemonic of the InputFields arrays. In another embodiment, the property name values can be compared with all relevant records in a database (e.g., Property Collections). For example, the compare mnemonic allows expressions to be built (e.g., a person's weight has to be greater than X, but less than Y, etc.). The control logic 300 can increment through all the InputFields, comparing the data value to the database using the mnemonic operator. In another example, the control logic 300 can determine if a prescription is appropriate for a patient, buy accessing the global patient record and pulling that data to run this qualification list on before making that determination. But beyond the search capability is the qualification of the search data to drive (or halt) additional processing, as discussed in more detail below. In another embodiment, the control logic can query a database to return matches of a property name value using the mnemonic value. The control logic then proceeds to step 312.

At step 312, the control logic 300 can determine whether matching values are found. If matching values are found, the control logic 300 proceeds to step 314. If matching values are not found, the control logic 300 proceeds to step 316.

At step 314, the control logic 300 can set the ‘result value’ to ‘True.’ The control logic 300 then proceeds to step 318.

At step 316, the control logic 300 can set the ‘result value’ to ‘False.’ The control logic 300 then proceeds to step 318.

At step 318, control logic 300 can determine the value of the ‘InvertResult’ property of the ‘QualificationList.’ The control logic 300 then proceeds to step 322.

At step 322, the control logic 300 can determine whether to invert the ‘result value.’ If the result is to be inverted, the control logic 300 continues to step 324. If the result is not to be inverted, the control logic 300 continues to step 326.

At step 324, the control logic 300 can invert ‘result value.’ In one embodiment, if a qualification fails and is set to ‘false,’ the control logic 300 can invert the value to ‘true,’ to drive additional processing. Since the control logic 300 can store the result as a result value in cache, the invert value can act as a gatekeeper by ensuring subsequent processing of the data. By way of example, if the client wants to qualify that a patient is “healthy enough” for a particular medication or regimen, certain parameters in the QualificationList can be run against the patient's record. If the control logic returns a ‘false’ value that the patient is not “healthy enough,” the processing would end. However, to continue subsequent analysis of the effectiveness of a drug, for example, the control logic can invert the ‘result value’ such that the “effectiveness” analysis can be run against the patient via additional parameters in the QualificationList to keep the analysis driving forward. The control logic 300 then proceeds to step 326.

At step 326, the control logic 300 can determine whether the ‘CacheResult’ property is ‘True.’ For example, the ‘CacheResult’ property can be true if the cache is static. If the ‘CacheResult’ property is ‘true,’ the control logic 300 proceeds to step 328. If the ‘CacheResult’ property is ‘false,’ the control logic 300 proceeds to step 332.

Add step 328, The control logic can cache ‘result value’ as the ‘qualification result.’ The control logic 300 then proceeds to step 330.

At step 330, the control logic 300 can return ‘result value’ as the result of the qualification. In one embodiment, the control logic 300 can generate and transmit a notification to the client that the ‘result value’ has been returned. The control logic 300 then terminates or awaits a new ‘QualificationList’ and can repeat the aforementioned steps.

At step 332, the control logic 300 can terminate qualification. The control logic 300 then terminates or awaits a new ‘QualificationList’ and can repeat the aforementioned steps.

In one embodiment, by way of example, the following diagram shows a ‘Property Collection’ called ‘Test’.

Test

| Property Name | Property Value |
|---|---|
| S1 | Circle |
| C1 | Orange |

The following is a non-limiting example of the ‘Qualification List’ control logic 300 for the ‘QL1’ ‘Qualification List’ against the ‘Test’ ‘Property Collection’:

The ‘QL1’ ‘Qualification List’ ‘Property Collection’ is consulted to determine if the ‘CacheResult’ property is ‘True’.

The value of the ‘CacheResult’ property is ‘True’, the ‘Qualification List’ processing logic looks in cache to determine if there is a ‘qualification result’ for ‘QL1’.

The ‘Qualification List’ processing logic determines that there is no cache value so processing continues.

The XPP of the first ‘InputField’ ‘Property Collection’ is defined as ‘Test.S1’. When this ‘execution context’ is used to retrieve the value for this XPP the following occurs:
The ‘execution context’ parses the ‘Test.S1’ XPP and determines that the ‘Property Collection’ is called ‘Test’.
The ‘execution context’ then determines if a ‘Property Collection’ called ‘Test’ exists in its ‘Property Collection Index’.
The ‘execution context’ determines that a ‘Property Collection’ called ‘Test’ does exist and retrieves a ‘reference’ to the ‘Property Collection’.
The ‘execution context’ then determines the ‘property path’ of the ‘Test.S1’ XPP.
The ‘execution context’ determines that the ‘property path’ is ‘S1’.
The ‘execution context’ then performs a ‘get’ operation on the ‘Test’ ‘Property Collection’ to retrieve the value of the ‘S1’ property.
The ‘Test’ ‘Property Collection’ returns a value of ‘Circle’.
The ‘qualification list processing’ then saves this value and associates it with the first input field.

The XPP of the second ‘InputField’ ‘Property Collection’ is defined as ‘Test.C1’. When this ‘execution context’ is used to retrieve the value for this XPP the following occurs:
The ‘execution context’ parses the ‘Test.C1’ XPP and determines that the ‘Property Collection’ is called ‘Test’.
The ‘execution context’ then determines if a ‘Property Collection’ called ‘Test’ exists in its ‘Property Collection Index’.
The ‘execution context’ determines that a ‘Property Collection’ called ‘Test’ does exist and retrieves a ‘reference’ to the ‘Property Collection’.
The ‘execution context’ then determines the ‘property path’ of the ‘Test.C1’ XPP.
The ‘execution context’ determines that the ‘property path’ is ‘C1’.
The ‘execution context’ then performs a ‘get’ operation on the ‘Test’ ‘Property Collection’ to retrieve the value of the′C1′ property.
The ‘Test’ ‘Property Collection’ returns a value of ‘Orange.
The ‘qualification list processing’ then saves this value and associates it with the second input field.

The ‘Qualification List’ processing logic uses the two input values and searches the ‘QualificationItems’ array looking for a ‘QualificationItem’ ‘Property Collection’ that contains a ‘Shape’ property value, which is equal (EQ) to ‘Circle’ and a ‘Color’ property value, which is equal (EQ) to ‘Orange’.

The ‘Qualification List’ processing does not find a ‘QualificationItem’ with these values and sets its ‘result value’ to ‘false’.

The ‘Qualification List’ processing logic then consults the ‘QL1’ ‘Qualification List’ ‘Property Collection’ to determine the value of the ‘InvertResult’ property.

The value of the ‘InvertResult’ property is ‘False’, so the ‘Qualification List’ processing logic

The ‘Qualification List’ processing logic then consults the ‘QL1’ ‘Qualification List’ ‘Property Collection’ to determine the value of the ‘CacheResult’ property and.

The value of the ‘CacheResult’ property is ‘True’, so the ‘Qualification List’ processing logic caches the ‘result value’ as the ‘qualification result’ for the ‘QL1’ ‘Qualification List’.

The ‘Qualification List’ processing logic returns the ‘result value’ as the ‘result’ of the qualification of QL1.

Although one or more embodiments may reference a patient, the present disclosure applies to any type of entity, whether a person, patient, customer, company, or other suitable entity capable of having data stored in a record associated with that entity. Similarly, although certain embodiments may reference electronic health records, the systems, methods, and concepts disclosed herein are equally applicable to any storage system or record type.

Persons skilled in the art will readily understand that advantages and objectives described above would not be possible without the particular combination of computer hardware and other structural components and mechanisms assembled in this inventive system and described herein. Additionally, the algorithms, methods, and processes disclosed herein improve and transform any general-purpose computer or processor disclosed in this specification and drawings into a special purpose computer programmed to perform the disclosed algorithms, methods, and processes to achieve the aforementioned functionality, advantages, and objectives. It will be further understood that a variety of programming tools, known to persons skilled in the art, are available for generating and implementing the features and operations described in the foregoing. Moreover, the particular choice of programming tool(s) may be governed by the specific objectives and constraints placed on the implementation selected for realizing the concepts set forth herein and in the appended claims.

The description in this patent document should not be read as implying that any particular element, step, or function can be an essential or critical element that must be included in the claim scope. Also, none of the claims can be intended to invoke 35 U.S.C. § 112(f) with respect to any of the appended claims or claim elements unless the exact words “means for” or “step for” are explicitly used in the particular claim, followed by a participle phrase identifying a function. Use of terms such as (but not limited to) “mechanism,” “module,” “device,” “unit,” “component,” “element,” “member,” “apparatus,” “machine,” “system,” “processor,” “processing device,” or “controller” within a claim can be understood and intended to refer to structures known to those skilled in the relevant art, as further modified or enhanced by the features of the claims themselves, and can be not intended to invoke 35 U.S.C. § 112(f). Even under the broadest reasonable interpretation, in light of this paragraph of this specification, the claims are not intended to invoke 35 U.S.C. § 112(f) absent the specific language described above.

The disclosure may be embodied in other specific forms without departing from the spirit or essential characteristics thereof. For example, each of the new structures described herein, may be modified to suit particular local variations or requirements while retaining their basic configurations or structural relationships with each other or while performing the same or similar functions described herein. The present embodiments are therefore to be considered in all respects as illustrative and not restrictive. Accordingly, the scope of the inventions can be established by the appended claims rather than by the foregoing description. All changes which come within the meaning and range of equivalency of the claims are therefore intended to be embraced therein. Further, the individual elements of the claims are not well-understood, routine, or conventional. Instead, the claims are directed to the unconventional inventive concept described in the specification.

What is claimed is:

1. A system for electronic health record data qualification, comprising:
a memory storing one or more property collections; and
a processor operably coupled to the memory and capable of executing machine-readable instructions to perform program steps, the program steps comprising:
receiving a qualification list in a first format from a client, the qualification list including at least one input field array having a property name, a property name value, a mnemonic, and a mnemonic value, an invert result property and an invert result property value;
query a database to return matches of a property name value using the mnemonic value;
setting a result value to true to indicate that a match was found;
determining the invert result value is true and inverting the result value.

2. The system of claim 1, the program steps further comprising converting the qualification list from the first format into a Property Collection format.

3. The system of claim 1, the program steps further comprising retrieving a property name value and a mnemonic value from the qualification list for all input field arrays.

4. The system of claim 1, wherein the database can be the qualification items array.

5. The system of claim 1, wherein the database can be a Property Collection.

6. The system of claim 1, the program steps further comprising determining a cache result property is False and terminating the qualification.

7. The system of claim 1, the program steps further comprising determining a cache result property is True and caching the result value as the qualification result.

8. The system of claim 1, the program steps further comprising returning the result value as the result of the qualification.

9. The system of claim 1, wherein the processor addresses data using an execution context property path (XPP).

10. The system of claim 1, wherein the at least one input field array also has an XPP property and an XPP value to retrieve the current value of the input field.

11. The system of claim 10, wherein the XPP value is an ASCII string.

12. A method of electronic health record data qualification, comprising:
receiving, via a processor, a qualification list in a first format from a client, the qualification list including at least one input field array having a property name, a property name value, a mnemonic, and a mnemonic value, an invert result property and an invert result property value;
querying a database to return matches of a property name value using the mnemonic value;
setting a result value to true to indicate that a match was found;
determining the invert result value is true and inverting the result value.

13. The method of claim 12, further comprising converting the qualification list from the first format into a Property Collection format.

14. The method of claim 12, further comprising retrieving a property name value and a mnemonic value from the qualification list for all input field arrays.

15. The method of claim 12, wherein the database can be the qualification items array.

16. The method of claim 12, wherein the database can be a Property Collection.

17. The method of claim 12, further comprising determining a cache result property is True and caching the result value as a qualification result.

18. The method of claim 12, further comprising returning the result value as the result of the qualification.

19. The method of claim 12, wherein the processor addresses data using an execution context property path (XPP).

20. The method of claim 12, wherein the at least one input field array also has an XPP property and an XPP value to retrieve the current value of the input field.
